(19) United States
(12) Patent Application Publication
(10) Pub. No.: US 2022/0287682 A1
(43) Pub. Date: Sep. 15, 2022

(54) Automatic Identification, Notification And Guidance To Regions Of Interest In Ultrasound Images On Devices With Limited Display Area

(71) Applicant: GE Precision Healthcare LLC, Wauwatosa, WI (US)

(72) Inventors: Arun Kumar Siddanahalli Ninge Gowda, Bangalore (IN); Srinivas Koteshwar Varna, Bangalore (IN)

(21) Appl. No.: 17/198,668

(22) Filed: Mar. 11, 2021

(51) Int. Cl. A61B 8/00; A61B 8/08

(52) U.S. Cl. CPC A61B 8/463; A61B 8/56; A61B 8/469; A61B 8/52

(57) ABSTRACT

An imaging system and method for operating the system to display images generated by the imaging system is capable of presenting the images on the display of a remote device along with various markers and/or indications in the displayed or viewable portion of the image that provide information on the location(s) of and direction(s) to any cropped portions or areas of the image not shown in the displayed portion, as well as any ROIs in the image displayed, but disposed in cropped areas of the image. The indications provide the user with information regarding the presence and location of these items, as well as other relevant information, such that the user can operate the remote device, such as by manipulating the display screen in a known manner, to move the areas of the image containing the selected item onto the display screen or area of the remote device.

### BACKGROUND OF THE INVENTION

The invention relates generally to imaging systems, and more particularly to structures and methods of displaying images generated by the imaging systems.

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device operably connected to the workstation or device.

With the advent of portable ultrasound machines, as well as the increased capability of remote device to display complex images with high resolutions, such as ultrasound and other imaging system images, users of ultrasound and other imaging systems are using their remote/mobile devices like smartphones and tablet devices to pair and work with ultrasound imaging system hardware.

However, the display size area and shape aspect ratio of these mobile devices varies among different devices, and all differ significantly from the size and shape/aspect ratio of the display area of traditional ultrasound imaging systems. More specifically, the display for an ultrasound imaging system has a large display area that is able to present the entire ultrasound image. In contrast, the remote/mobile devices have a much smaller display area in which to fit the ultrasound image.

While the ultrasound image can be readily sent to and displayed on the screen of the remote device, the formatting of the ultrasound image is retained when sent to the remote device. The primary issue with the smartphones/tablets in displaying the entire image is their relatively smaller display size and higher aspect ratio (typically >1.3) compared to the displays associated with traditional ultrasound scanners. Hence when the entire ultrasound image from the scanner, which predominantly has an aspect ratio closer to 1.0, and is closer to square image format, is displayed on these type of remote devices, the image will be too small to perform diagnosis and obtain the relevant information. This is particularly a major problem when the remote device is used in Portrait mode.

As such, the remote device is often incapable of displaying the entire image within the display for the remote device without zooming out on the image, which effectively reduces the resolution for the image to an unusable extent. In order to overcome this limitation, usually the ultrasound image is resized either manually or automatically, and is displayed on the screen such that the height of the ultrasound image, which corresponds to the depth of the scanned image, fits within the entire height of the display. In such a case, while the user can view most of the central portions of the image on display with finer details being viewable, regions of the ultrasound image on either side will not be visible to the user.

More specifically, when the system or user zooms in on the image to increase the size of the image on the remote device to achieve the proper resolution for viewing the image in a live scanning or offline mode, the ultrasound image on the display of the remote device is cropped, such that the entire ultrasound image is not presented on the screen of the remote device. This cropping of the ultrasound image can prevent the individual from seeing any regions of interest (ROIs) which include, but are not limited to, organs-structures, or anomalies or other regions of clinical relevance or other artifacts like needles (used during a procedure) present in the portions of the ultrasound image not presented on the remote device screen. This is particularly an issue when the image is sent to the mobile or remote device in a manner that initially presents the image in a cropped manner, such that the user may believe the entire image is being displayed and consequently not be aware of the existence of the cropped portions of the displayed image.

Therefore, it is desirable to develop a system and method for the representation of ROIs in the ultrasound image presented on a screen of a remote device that notifies the user of the presence of any ROIs within cropped areas of the ultrasound image not shown on the remote device screen as well as directions to navigate within the image in the remote device screen to those ROIs. In addition, it is also desirable to have a system and method that provides indications to the user on the presence/existence of cropped regions of the ultrasound image that are currently not being displayed on screen

### BRIEF DESCRIPTION OF THE DISCLOSURE

In the present disclosure, an imaging system and method for operating the system to display images generated by the imaging system is capable of presenting the images on the display of a remote device along with various markers and/or indications in the displayed portion of the image that provide information regarding aspects of the image that are not presented on the display of the remote device.

In one aspect, markers are presented on the remote device display screen concerning the location(s) of and direction(s) to any ROIs in the image displayed, but disposed in cropped areas of the image. The indications provide the user with information regarding the presence and location of these items, as well as other relevant information, such that the user can operate the remote device, such as by manipulating the display screen in a known manner, to move the areas of the image containing the selected item(s) onto the display screen of the remote device.

In another aspect, indications are provided on the remote device display screen that illustrate the presence of cropped portions of the image presented on the remote device display screen. These indications are provided regardless of the presence of any markers that may additionally be necessary to provide location(s)and direction(s) to any ROIs in the cropped portions or areas of the displayed image. The indications for the cropped portions provide notice to the user of the cropped portions such that the user can shift the image on the remote device display to present the cropped portion(s).

According to one exemplary aspect of the disclosure, an imaging system for displaying images obtained by the imaging, system on a display of a remote device including an imaging probe adapted to obtain image data on an object to be imaged, a processor operably connected to the probe to form an image from the image data, and a display operably connected to the processor for presenting the image on the display, wherein the processor is configured to determine a viewable portion and one or more cropped portions of the image to be presented on the display, to implement an algorithm to identify a location of at least one region of interest (ROI) in one or more of the cropped portions of the image and to provide at least one ROI marker associated with the at least one ROI in the one or more cropped portions in the viewable portion of the image on the display.

According to another exemplary aspect of the disclosure, a method for displaying an image obtained by an imaging system on a display of a remote device includes the steps of determining a viewable portion and one or more of cropped portions of the image to be displayed on the remote device, implementing an algorithm to identify a location of at least one region of interest (ROI) in one or more of the cropped portions of the image, providing at least one ROI marker associated with the at least one ROI in the one or more cropped portions in the viewable portion of the image and presenting the viewable portion and the at least one ROI marker on the display of the remote device.

It should be understood that the brief description above is provided to introduce in simplified form a selection of concepts that are further described in the detailed, description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a schematic block diagram of an imaging system formed in accordance with an embodiment.

FIG. 2 is a schematic block diagram of an imaging system thrilled in accordance with another embodiment.

FIG. 3 is a flowchart of a method for operating the imaging system shown in FIG. 2 in accordance with an embodiment.

FIG. 4 is a schematic view of an ultrasound image and indications presented on a display screen of a remote device in accordance with an embodiment.

FIG. 5 is a schematic view of an ultrasound image and indications presented on a display screen of a remote device in accordance with another embodiment.

FIG. 6 is a schematic view of the manipulation of the ultrasound image of FIG. 4 on the remote device display screen using the indications in accordance with an embodiment.

FIG. 7 is a schematic view of an ultrasound image from a linear probe and indications presented on a display screen of a remote device in accordance with an embodiment.

FIG. 8 is a schematic view of an ultrasound image from a linear probe and indications presented on a display screen of a remote device in accordance with another embodiment.

### DETAILED DESCRIPTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. One or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Although the various embodiments are described with respect to an ultrasound imaging system, the various embodiments may be utilized with any suitable imaging system, for example, X-ray, computed tomography, single photon emission computed tomography, magnetic resonance imaging, or similar imaging systems.

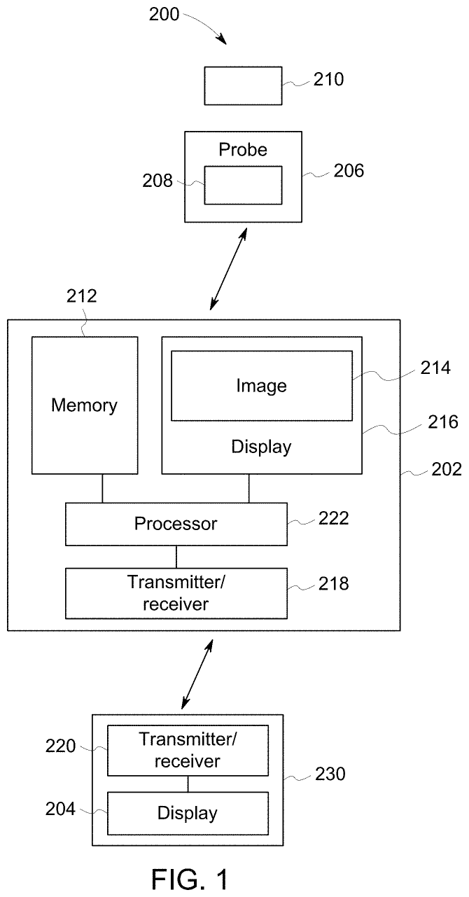

FIG. 1 is a schematic view of an imaging system 200 including an ultrasound imaging system 202 and a remote device 230. The remote device 230 may be a computer, tablet-type device, smartphone or the like that can be an off-the-shelf device, or a device dedicated for use as the remote device 230 in conjunction with the imaging system 202. The term “smart phone” as used herein, refers to a portable device that is operable as a mobile phone and includes a computing platform that is configured to support the operation of the mobile phone, a personal digital assistant (PDA), and various other applications. Such other applications may include, for example, a media player, a camera, a global positioning system (GPS), a touchscreen, an interact browser. Wi-Fi, etc. The computing platform or operating system may be, for example, Google Android™, Apple iOS™, Microsoft Windows™, Blackberry™, Linux™, etc. Moreover, the term “tablet-type device” refers to a portable device, such as for example, a Kindle™ or iPad™. The remote device 230 may include a touchscreen display 204 that functions as a user input device and a display. The remote device 230 communicates with the ultrasound imaging system 202 to display an image 214 based on image data acquired by the ultrasound imaging system 202 on the display 204. The remote device 230 also includes any suitable components for image viewing, manipulation, etc., as well as storage of information relating to the image 214.

A probe 206 is in communication with the ultrasound imaging system 202. The probe 206 may be mechanically coupled to the ultrasound imaging system 202. Alternatively, the probe 206 may wirelessly communicate with the imaging system 202, The probe 206 includes transducer elements/an array of transducer elements 208 that emit ultrasound pulses to an object 210 to be scanned, for example an organ of a patient. The ultrasound pulses may be back-scattered from structures within the object 210, such as blood cells or muscular tissue, to produce echoes that return to the transducer elements 208. The transducer elements 208 generate ultrasound image data based on the received echoes. The probe 206 transmits the ultrasound image data to the ultrasound imaging system 202 operating the imaging system 200. The image data of the object 210 acquired using the ultrasound imaging system 202 may be two-dimensional or three-dimensional image data. In another alternative embodiment, the ultrasound imaging system 202 may acquire four-dimensional image data of the object 210. In generating the image 214, the processor 222 is also configured to automatically identify regions of interest (ROIs) 224 within image 214, and to provide identifications of those ROIs 224 within the image 214.

The ultrasound imaging system 202 includes a memory 212 that stores the ultrasound image data. The memory 212 may be a database, random access memory, or the like. A processor 222 accesses the ultrasound image data from the memory 212. The processor 222 may be a logic based device, such as one or more computer processors or microprocessors. The processor 222 generates an image based on the ultrasound image data. After formation by the processor 222, the image 214 is presented on a display 216 for review, such as on display screen of a cart-based ultrasound imaging system 202 having an integrated display/monitor 216, or an integrated display/screen 216 of a laptop-based ultrasound imaging system 200, optionally in real time during the procedure or when accessed after completion of the procedure.

In one exemplary embodiment, the ultrasound imaging system 202 can present the image 214 on the associated display/monitor/screen 216 along with a graphical user interface (GUI) or other displayed user interface. The image 214 may be a software based display that is accessible from multiple locations, such as through a web based browser, local area network, or the like. In such an embodiment, the image 214 may be accessible remotely to be displayed on a remote device 230 in the same manner as the image 214 is presented on the display/monitor/screen 216.

The ultrasound imaging system 202 also includes a transmitter/receiver 218 that communicates with a transmitter/receiver 220 of the remote device 230. The ultrasound imaging system 202 and the remote device 230 may communicate over a direct peer to peer wired/wireless connection or a local area network or over an internet connection, such as through a web-based browser.

An operator may remotely access imaging data stored on the ultrasound imaging system 202 from the remote device 230. For example, the operator may log onto a virtual desktop or the like provided on the display 204 of the remote device 230. The virtual desktop remotely links to the ultrasound imaging system 202 to access the memory 212 of the ultrasound imaging system 202. Once access to the memory 212 is obtained, the operator may select image data to view. The image data is processed by the processor 222 to generate an image 214. For example, the processor 222 may generate a DICOM image 214. The ultrasound imaging system 202 transmits the image 214 to the display 204 of the remote device 230 so that the image 214 is viewable on the display 204.

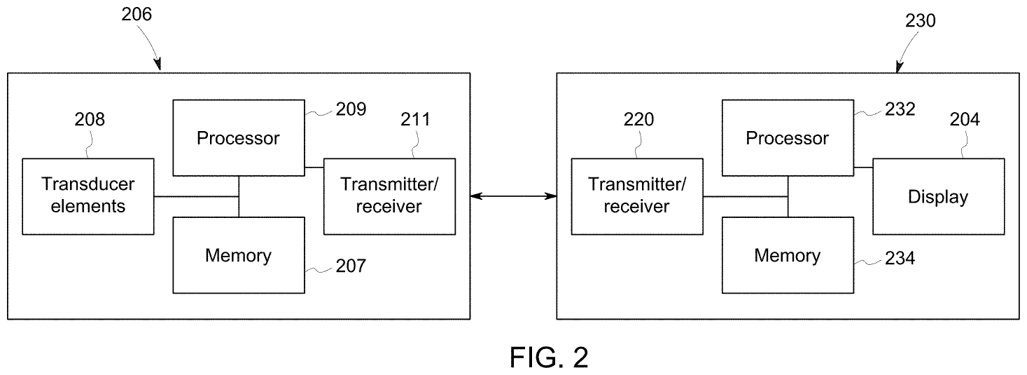

Looking now at FIG. 2, in an alternative embodiment, the imaging system 202 is omitted entirely, with the probe 206 constructed to include memory 207, a processor 209 and transceiver 211 in order to process and send the ultrasound image data directly to the remote device 230 via a wired or wireless connection. The ultrasound image data is stored within memory 234 in the remote device 230 and processed in a suitable manner by a processor 232 operably connected to the memory 234 to create and present the image 214 on the remote display 204.

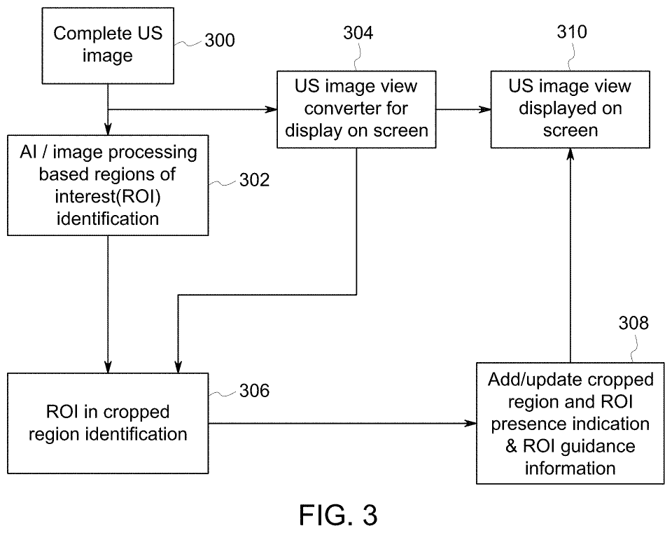

In either embodiment, the image 214 generated by the processor 222,232 is formatted for presentation on the display 216 for the imaging system 202, and this formatting is retained in the embodiment where the image 214 is transmitted to the remote device 230 or when generated on the remote device 230 for display in a portrait or landscape format. In order to effectively display the image 214 on the display 204 of the remote device 230, in the method of FIG. 3, after the creation of the image 214 by the processor 222,232 in block 300, in block 302 the processor 222,232 also ascertains the locations of regions of interest (ROIs) 224 which include, but are not limited to, organs/structures, or anomalies 226 or other regions of clinical relevance or other artifacts like needles (used during a procedure) present within the image 214 using known identification processes and/or algorithms for ultrasound or other imaging system image generation. For example, traditional image processing techniques, or Artificial Intelligence (AI) based—approaches including machine learning (ML) and deep learning (DL), among others, or a combination of both can be used to identify and localize these ROIs. 224 and/or anomalies 226 within the image 214. For AI based identification approaches the end goal of identifying and localizing these ROIs 224/anomalies 226 could be formulated as either image segmentation or object localization problem. Though ML based approaches like support vector machines (SVM), random forest (RF), etc., can be used to solve these problems, convolutional neural networks (CNN), a class of DL based models, are best suited for such tasks yielding much better accuracy and adaptability across various imaging conditions.

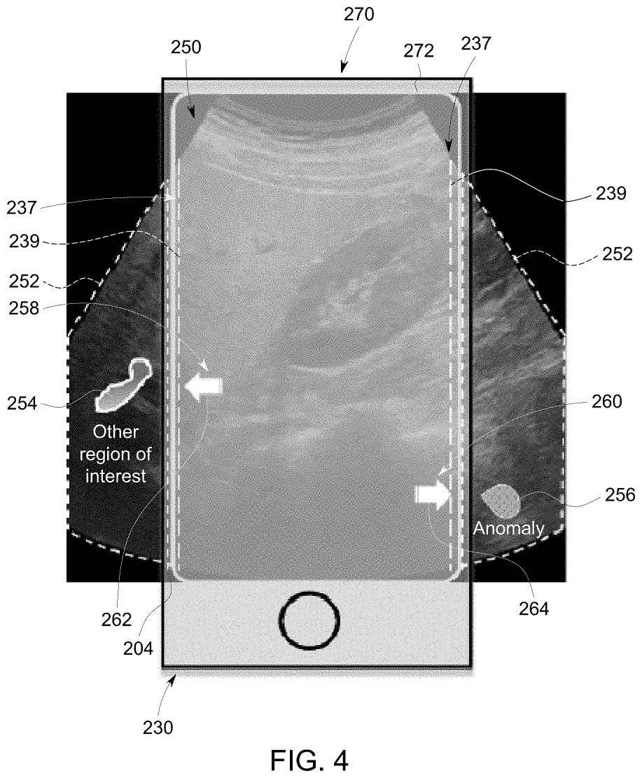

In block 304 the processor 222,232 additionally determines and converts the image 214 into a proper 1brmat for the image 214, such as from a first display format to a second display format, from an imaging system display format to a remote device display format, from a landscape format to a portrait format, or vice versa, and/or determines the magnification of the image 214 required to effectively present the image 214 on the remote device display 204. As in an exemplary embodiment the image 214 is formatted for presentation on the display 216 in a generally square configuration, the size, aspect ratio and shape of the remote device display 204 will result in only a portion of the image 214 being presentable on the remote device display 204 with the appropriate format conversion and/or magnification. In performing this analysis, the processor 222,232 determines what portion(s)s 250 (FIG. 4) of the image 214 will be viewable on the remote device display 204 and what portion(s) 252 (FIG. 4) of the image 214 will be cut off or cropped on the remote device display 204.

Using the information on the location of the ROIs 224 and anomalies 226 in the image 214 from block 302 and the information regarding the viewable image areas) 250 and cut off or cropped image portion(s) or area(s) 252 of the image 214 from block 304, in block 306 the processor 222,232 can locate the positions of the ROIs 224 and the anomalies 226 in the viewable image portion(s) 250 and cropped image portion(s) 252. For those ROIs 224 and anomalies 226 located in the viewable image portion(s) 250, the processor 222,232 provides suitable indications 254, 256 identifying the ROIs 224 and anomalies 226 in the viewable image portion(s) 250. Further, the processor 222,232 also provides a marker or indication 237, e.g., dotted lines 239, extending along the edges of the display 204 that identify those edges of the viewable portion 250 of the image 214 on the display 204 that have cropped image portion(s) 252 extending past the associated edge of the display 204.

With regard to the ROIs 224 and anomalies 226 identified by the processor 222,232 and located in the cropped image portion(s) 252, the processor 222,232 also provides suitable indications 254, 256 identifying the ROIs 224 and anomalies 226 in the cropped image portion(s) 252 in block 306. However, as the indications 254,256 are not readily viewable on the remote device display 204, in block 308 the processor 222,232 modifies the viewable image portion(s) 250 to include one or more markers 258,260 which are subsequently presented on the remote device display 204 along with the viewable portion 250 of the image 214 in block 310.

The markers 258,260 are readily seen within the viewable image portion 250 and provide an indication within the viewable image portion(s) 250 of the presence and location of an ROI 224 and/or an anomaly 226 in one or more of the cropped image portions 252. The markers 258, 260 can have any suitable form, and any suitable shape and/or color, and in the illustrated exemplary embodiment of FIG. 4, take the form of arrows 262,264. The arrows 262,264 are illustrated in different manners, e.g., colors, to correspond to the type of the region of interest they are associated with, which could be ROI 224 or anomaly 226 as identified in the image 214. The arrows 262,264 are located in the viewable image portion 250 in alignment with the location of the associated ROI 224 and/or anomaly 226 in the associated cut off or cropped image portion 252, with the arrow 262,264 pointing in the direction of the location of the associated ROI 224 and/or anomaly 226. The markers 258,260 can additionally be aligned with the ROI 224/anomaly 226 in a depth direction where the position of the marker 258,260 in the viewable image portion 250 corresponds to the depth of the ROI 224/anomaly 226 in the associated cropped portion 252. In another exemplary embodiment, the other attributes of the indicators 254,256, markers 258,260 and/or arrows 262,264 can be altered to provide additional information on the associated ROI 224 and/or anomaly 226. For example, the marker 258,260 can function as a link when selected to display a note or other written or typed information regarding the ROI 224/anomaly 226 associated with the marker 258,260, or to shift the viewable image portion 250 to present the WA 224/anomaly 226 associated with the marker 258,260 in the viewable display portion 250. The indicators 254,256, markers 258,260 and/or arrows 262,264 can also be made to have different shapes corresponding to the types of ROI 224 or anomaly 226 identified by the indicators 254,256, markers 258,260 and/or arrows 262,264. Further, the marker 258,260 can intermittently flash, or include other indicia thereon or associated therewith, such as when the marker 258,260 can be activated as a link to display the indicia on the display 204, to indicate the significance of the ROI 224/anomaly 226 associated with the marker 258,260. Also, the size of the marker 258,260, such as the size of the head of the arrow 262,264 and/or the length of the tail of the arrow 262,264 can provide information on the distance of the associated ROI 224/anomaly 226 in the cropped portion 252 from the viewable image portion 250.

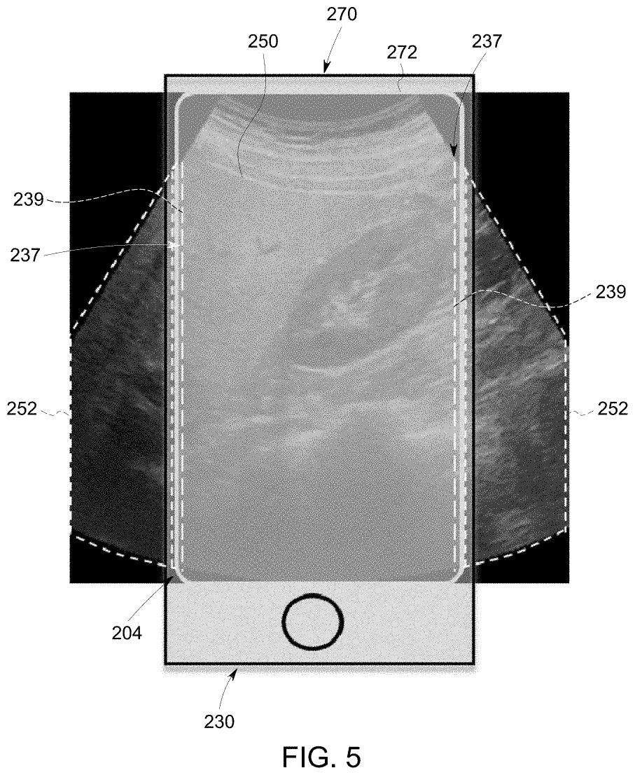

Referring now to FIG. 5, in the situation where the image 214 presented on the display screen 204 does not contain any ROIs 224 or anomalies 226, the image 214 is still presented with the markers or indications 237 for the identification of any cropped portions 252 of the image 214.

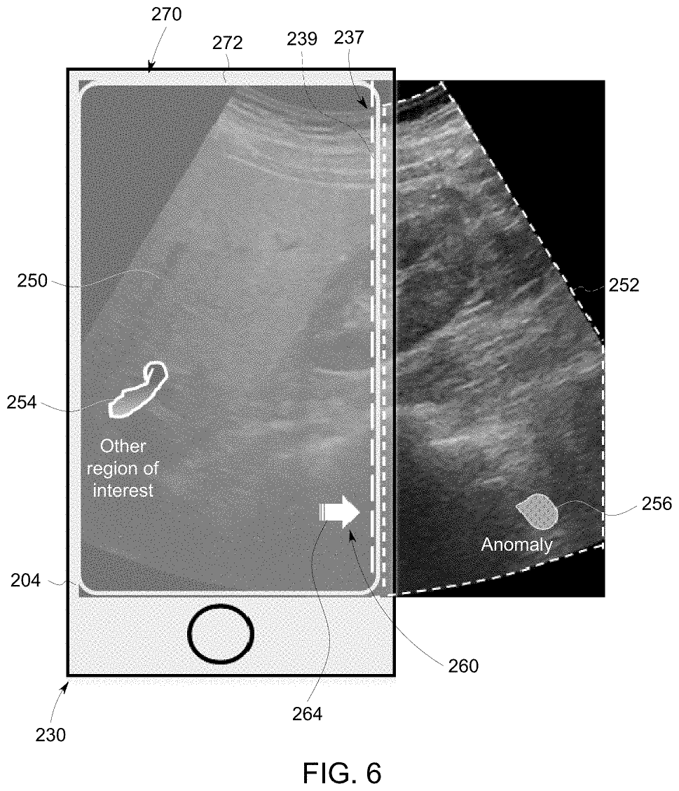

Looking now at FIG. 6, once the viewable image portion 250 is presented on the remote device display 204, the user can manipulate the image 214 using a suitable user input 270 for the remote device 230, which can be any suitable input device including a keyboard, mouse, or in the illustrated exemplary embodiment of FIG. 6, a touch screen interface 272 that enables the user to shift the viewable portion 250 by swiping the viewable portion 250 on the interface 272, in order to shift the area of the image 214 forming the viewable image portion 250. In this embodiment, as the user contacts the touch screen interface 272, the user swipes their finger along the screen interface 272 in a direction they wish to move the viewable image portion 250. Thus, by moving a finger to the right on the interface 272, the viewable image portion 250 shifts to the right, such that the cropped portion 252 located to the left of the viewable image portion 250 is moved into the viewable image portion 250. Further, by contacting the screen interface 272 and making pinching or expanding movements with two fingers, the user can increase or decrease the magnification of the viewable image portion 250.

Alternatively, the user can operate the remote device 230 to shift the image 214 on the remote device display 204 utilizing the user input 270 in a manner similar to that disclosed in U.S. Pat. No. 7,274,377, entitled Viewport Panning Feedback System, the entirety of which is expressly incorporated herein by reference for all purposes. In this manner, the user can shift or navigate the image 214 relative to the remote device display 204 to enable the user to position the ROIs 224 and/or anomalies 226 in any cut off or cropped image portion 252 within the viewable image portion 250 presented on the remote device display 204. The markers 258,260 enable the user to quickly locate and view the associated ROIs 224 and/or anomalies 226 in the cropped portions 252 due to the information provided by the markers 258,260, e.g., information on the presence and location of the associated ROI 224 or anomaly 226.

In addition, while the user navigates to the ROI 224 or anomaly 226 associated with the marker 258,260, the marker 258,260 can remain within the viewable portion 250 in alignment with the ROI 224 or anomaly 226 until the ROI 224 or anomaly 226 is disposed within the viewable portion 250 of the image 214 on the remote device display 204. Also, as the user navigates to the ROI 224 or anomaly 226, the marker 258,260 can be altered corresponding to the changes made in the position of the viewable portion 250 on the display 204 relative to the cropped portion 252 containing the ROI 224 or anomaly 226. For example, the size of the marker 258,260 can increase or decrease in size corresponding to the user shifting the viewable portion 250 on the display 204 away from or towards the ROI 224 or anomaly 226, e.g., the marker 258,260 gets larger as the viewable portion 250 is shifted towards the ROI 224/anomaly 226 and smaller as the viewable portion 250 is shifted away from the ROI 224/anomaly 226. Further, as the viewable portion 250 of the image 214 is shifted on the display screen 204, the markers or indications 237 for the cropped portions 252 Shift along with the image 214 and change their length and position along the edges depending on the shape/extent of cropped regions still present along those edges, and disappears entirely when the viewable image portion 250 does not have any cropped portions 252 in the direction of the particular edge of the display 204.

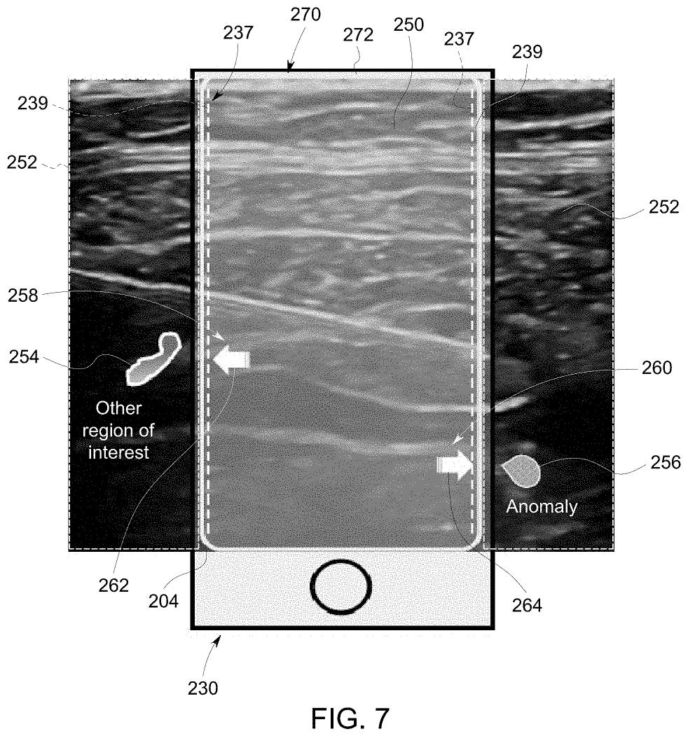

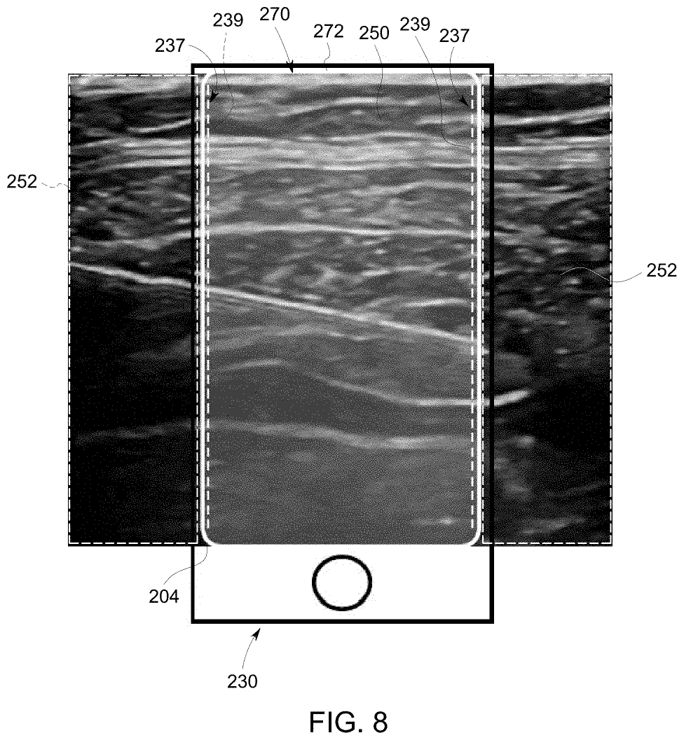

Looking now at FIGS. 7 and 8, the display 204 including the ROI 224, anomaly 226, indications 237, and/or markers 258,260 is also applicable when the format for the image 214 is shifted. For example, instead of the curved image view of FIGS. 4-6 provided by a convex/sector probe 206, when a linear probe 206 is utilized, the resulting image 214 is formatted as shown in FIGS. 7 and 8. In this format, the image 214 is still presented on the display 204 with any applicable markers 258,260 for an ROI 224, and/or an anomaly 226, as well as the markers or indicators 237, even if no markers 258,260 are present in the display 204, as shown in FIG. 8, and as described with respect to the prior embodiments. The image 214 can be shifted by the user on the display 204 to navigate to and present the ROIs 224 and/or anomalies 226, if present in the cropped portions 252, within the viewable image portion 250, or simply to display the cropped portions 252 identified by the markers or indications 237 on the display 204.

In other alternative embodiments, the capabilities of the remote device 230 can enable the user to zoom in and out on the image 214 to increase or decrease the size of the viewable image portion/area 250 in any known manner, Further, the image 214 being presented on the remote device display 204 can he a recorded and/or stored image, a real time image, a three dimensional or volumetric image, or a video stream, or any suitable combination thereof. In another exemplary embodiment, the markers 258,260 can be employed as selectable links or icons on the remote device 230 that when activated on the remote device display/screen 204 by the user, automatically navigates and/or shifts the image 214 to present the ROI 224 and/or anomaly 226 associated with the selected marker 258,260 in the viewable portion/area 250.

The written description uses examples to disclose the invention, including; the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for displaying an image obtained by an imaging system on a display of a remote device, the method comprising the steps of:
determining a viewable portion and one or more cropped portions of the image to be displayed on the remote device;
implementing an algorithm to identify a location of at least one region of interest (ROI) in one or more of the cropped portions of the image;
providing at least one ROI marker associated with the at least one ROI in the one or more cropped portions in the viewable portion of the image; and
presenting the viewable portion and the at least one ROI marker on the display of the remote device.

2. The method of claim 1, wherein the step of determining the viewable portion and the one or more of cropped portions of the image comprises:
comparing an imaging system display format with a remote device display format; and
converting the image from the imaging system display format the remote device display format.

3. The method of claim 1, further comprising the step of providing at least one cropped portion marker in the viewable portion of the image associated with the one or more cropped portions.

4. The method of claim 3, wherein the step of providing the at least one cropped portion marker comprises
placing the at least one cropped portion marker along an edge of the viewable portion adjacent to the one or more cropped portions.

5. The method of claim 1, wherein the step of providing the at least one ROI marker comprises:
placing the at least one ROI marker in the viewable portion along an edge of the viewable portion adjacent to each of the one or more cropped portions.

6. The method of claim 1, wherein the step a providing at least one ROI marker comprises the step of:
placing the at least one ROI marker in the viewable portion in alignment with the location for the associated at least one ROI in the one or more cropped portions.

7. The method of claim 1, wherein the at least one ROI marker includes information regarding the at least one ROI.

8. The method of claim 7, wherein the at least one ROI marker includes information on a distance of the at least one ROI in the one or more cropped portions from the viewable portion.

9. The method of claim 8, wherein information on the distance of the at least one ROI in the one or more cropped portions from the viewable portion is indicated by a size of at least a portion of the at least one ROI marker.

10. The method of claim 7, wherein the at least one ROI marker includes information on a type of the at least one ROI in the one or more cropped portions.

11. The method of claim 10, wherein information on the type of the at least one ROI in the one or more cropped portions is indicated by a color of the at least one ROI marker.

12. The method of claim 1, further comprising the step of shifting the viewable portion on the remote device display to shift one of the one or more cropped portions and the at least one ROI into the viewable portion after presenting the viewable portion and at least one ROI marker on the remote device display.

13. The method of claim 12, wherein the step of shifting the viewable portion on the remote device display comprises interacting with the remote device.

14. The method of claim 13, wherein the remote device display is a touch screen, and wherein the step of interacting with the remote device comprises swiping the viewable portion on the touch screen.

15. An imaging system for displaying images obtained by the imaging system on a display of a remote device, the imaging system comprising:
an imaging probe adapted to obtain image data on an object to be imaged;
a processor operably connected to the probe to form an image from the image data; and
a display operably connected to the processor for presenting the image on the display,
wherein the processor as configured to determine a viewable portion and one or more cropped portions of the image to be presented on the display, to implement an algorithm to identify a location of at least one region of interest (ROI) in the one or more of the cropped portions of the image and to provide at least one ROI marker associated with the at least one ROI in the one or more cropped portions in the viewable portion of the image on the display.

16. The imaging system of claim 15 wherein the processor is configured to determine the viewable portion and the one or more cropped portions of the image to be displayed on the display by comparing a first display format with second display format, wherein the second display format is associated with the display.

17. The imaging system of claim 15, wherein the at least one ROI marker includes information regarding the at least one ROI.

18. The imaging system of claim 15, further comprising:
a remote device, wherein the display is a remote device display, and wherein the at least one ROI marker is disposed in the viewable portion on the remote device display in alignment with the location for the associated at least one ROI in the one or more cropped portions.

19. The imaging system of claim 18, wherein the processor is disposed within the remote device and operably connected to the remote device display.

20. The imaging system of claim 19, wherein the probe is operably connected directly to the processor in the remote device.

21. The imaging system of claim 18, comprising:
an ultrasound imaging system,
wherein processor is disposed within the ultrasound imaging system;
wherein the probe is operably connected to the ultrasound imaging system, and
wherein the remote device is operably connected to the ultrasound imaging system.
